(19) United States
(12) Patent Application Publication
(10) Pub. No.: US 2024/0197948 A1
(43) Pub. Date: Jun. 20, 2024

(54) KEEP-CLEAN DEVICE

(71) Applicant: Virobuster International GmbH, Windhagen (DE)

(72) Inventor: Fehmi Yigit, Gronau (DE)

(73) Assignee: Virobuster International GmbH, Windhagen (DE)

(21) Appl. No.: 18/555,932

(22) PCT Filed: Mar. 21, 2022
(86) PCT No.: PCT/EP2022/057366

§ 371 Date: Oct. 18, 2023

(30) Foreign Application Priority Data
Apr. 28, 2021 (EP) 21170935.7

(51) Int. Cl. A61L 9/20

(52) U.S. Cl. CPC A61L 9/20; A61L 2209/134; A61L 2209/14

(57) ABSTRACT

The invention relates to a purification device for inactivating microorganisms, such as bacteria, germs, mold and/or viruses, present in a gaseous medium, in particular air, flowing through the purification device, with an irradiation device for UV irradiation, in particular UV-C irradiation, of the medium, wherein a guiding means flow-connected to the irradiation device is provided for supplying a partial flow of the flow of the medium flowing through the purification device to the irradiation device, wherein at least one further guiding means is provided for guiding a further partial flow and wherein the further guiding means does not open into the irradiation device and/or is guided past the irradiation device and/or is spatially separated and/or flow-separated from the irradiation device.

### CROSS-REFERENCE TO RELATED APPLICATIONS

The present application is the U.S. national stage application of international application PCT/EP2022/057366, filed Mar. 21, 2022, which international application was published on Nov. 3, 2022, as International Publication WO 2022/228779 A1. The international application claims priority to European Patent Application No. 21170935.7, filed Apr. 28, 2021. The international application and European application are hereby incorporated by reference herein in their entireties.

### FIELD

The invention relates to a purification device for inactivating and/or killing microorganisms, such as bacteria, germs, mold and/or viruses, present in a gaseous medium flowing through the purification device, in particular air and/or a gas mixture and/or a vapor mixture. The purification device has an irradiation device for UV irradiation, in particular UVC irradiation, of the medium.

In particular, the present invention relates to the technical field of so-called UV disinfection. The term “UV disinfection” refers to processes in which microorganisms—also referred to as microbes—can be killed by treatment with UV radiation. UV disinfection can be used for drinking water treatment, surface disinfection, exhaust air treatment, but also in the field of hygienic food processing or similar. Air in public areas can also be kept “clean” in this way, i.e. with a low level of harmful microorganisms.

### BACKGROUND

In the following, UV-C radiation is understood to be radiation with a wavelength between 100 nm and 280 nm, in particular UV-C radiation at a wavelength between 200 nm and 280 nm, preferably 240 nm to 280 nm, being used in the context of UV disinfection.

UV disinfection uses in particular a wavelength of UV radiation between 200 and 300 nm. The emitted UV radiation has a bactericidal effect—that is, it is absorbed by DNA and destroys its structure, inactivating living cells. In this way, microorganisms such as viruses, bacteria, yeasts and fungi can be rendered harmless with UV radiation within a very short time, especially within a few seconds.

With sufficiently high irradiance, UV disinfection is thus a reliable and ecological method, since in particular no addition of further chemicals is necessary. It is particularly advantageous that microorganisms cannot develop resistance to UV radiation. Finally, UV disinfection can also interrupt the reproduction of microorganisms.

The UV disinfection process can also be referred to as “Ultraviolet Germicidal Irritation” (UVGI) and/or microbial disinfection, in particular using UV radiation with a wavelength of 254 nm. The use of UV disinfection is particularly advantageous for virus inactivation in air purification, as it enables large volumes of air to be freed from viruses.

The outbreak of the Corona pandemic in early 2020 demonstrates the urgent need to have economical, efficient and ecological virus inactivation processes in place.

Air filtration on the basis of so-called HEPA filters is well known and catches particles such as viruses in particular. On the other hand, there are air disinfection devices where the particles, germs or VOC's (volatile organic compounds) are not intercepted, but treated by UV-C radiation ozone, ions or otherwise.

The main advantage of HEPA filters is that they can handle relatively large air volumes with low energy consumption and relatively simple mechanical and technical design. Air disinfection devices are bound to a certain exposure and/or treatment time to achieve sufficient effectiveness. This so-called dwell time limits the air volume capacity of the devices.

The aim of current developments is to provide devices that enable media streams with a large throughput, especially air streams, to be cleaned efficiently. Another or alternative goal is to also kill those particles that are comparatively resistant to UV radiation. Ultimately, various basic radiation doses are known, which may differ depending on the microorganisms to be killed.

The UV radiation dose (J/m²) is obtained by multiplying the UV irradiance (W/m²) and the UV irradiation time (s).

Ultimately, the inactivation of microorganisms depends in particular on the power or the achieved intensity of the UV radiation. The distance of the medium flow to the radiation source also has an influence on the efficiency of the disinfection and/or cleaning.

A problem in this context is that a sufficiently long residence time in the irradiation device and/or irradiation chamber must be ensured for efficient virus inactivation of the microorganisms. At the same time, a high “throughput” of the medium should also be achieved, so that in particular the total volume treated by the purification device and/or the total volume flow passed through the purification device should be as high as possible. For example, volume flows of at least 800 m³/h, preferably at least 1000 m³/h and in particular between 1100 to 2000 m³/h should be treated. However, an irradiation device designed for this volume flow is associated with very high costs—both in terms of operation and manufacture.

### SUMMARY

The object of the present invention is to provide an improved purification device for inactivating microorganisms present in a medium flowing through the purification device, in particular wherein a possible high throughput of the medium flowing through the purification device is to be achieved with simultaneous use of an irradiation device for UV irradiation.

The aforementioned object is solved according to the invention by a purification device for inactivating and/or killing microorganisms, such as bacteria, germs, mold and/or viruses, present in a gaseous medium flowing through the purification device, in particular air, a gas mixture and/or a vapor mixture, with an irradiation device for UV irradiation, in particular UV-C irradiation, of the medium according to claim 1. According to the invention, the purification device has a guiding means flow-connected to the irradiation device for supplying a partial flow of the flow of the medium flowing through the purification device to the irradiation device. In addition, in particular at least one further guiding means is provided for guiding a further, in particular non-irradiated, partial flow. The further guiding means may not open into the irradiation device and/or may be spatially separated and/or flow-separated from the irradiation device. Alternatively or additionally, it is provided that the further guiding means is guided past the irradiation device.

According to the invention, the possibility has now been created that with a high treated air volume, sufficient residence time for air disinfection can also be made possible at the same time.

Ultimately, it is particularly the case that at least two flow paths and/or flow ways are provided. The one flow path and/or flow way is formed by the partial flow guided into the irradiation device. This partial flow is ultimately treated in the irradiation device with UV radiation, in particular UV-C radiation. In addition, a further flow path and/or flow way is formed by the at least one further partial flow. This further partial flow is not treated in and/or guided through the irradiation device.

In particular, it is provided that the further guiding means can be routed around or past the irradiation device, in particular by the further guiding means being designed as a bypass line to the irradiation device. In particular, the further guiding means is separated from the guiding means in terms of flow and/or spatially separated from the guiding means.

Furthermore, only a (single) partial flow of the medium flowing through the purification device can be fed to the irradiation device via the guiding means, in particular by the guiding means being flow-connected to the irradiation device. The remaining flow of the medium flowing through the purification device can be guided in particular through the at least one further guide means in at least one further partial flow.

The aforementioned flow guidance according to the invention is associated with the advantage that the total flow guided through the purification device can be increased, wherein according to the invention only a part of the total flow has to be guided through the irradiation device. In the course of the invention, it has been found that, surprisingly, the treatment of a partial flow of the medium stream is sufficient to achieve a sufficiently high degree of purity—that is, a sufficiently high proportion of killed microorganisms—of the purified medium stream. According to the invention, this is justified by the fact that an improved efficiency of the inactivation can also be achieved by the increased total quantity and/or the increased total volume flow. In particular, according to the invention, an improved exchange of air is made possible, for example in closed rooms, which is accompanied by an increase in the volume flow treated in the hour. By means of the bypass line according to the invention and/or the at least one further guiding means, at least one (further) partial flow can be provided in the purification device, which is not treated by the irradiation device, but which is mixed with the volumetric flow treated in the irradiation device before or at the exit from the purification device. According to the invention, the resulting “mixed flow” continues to have a high purity content and/or ensures the requirements placed on an air treatment device. If the entire higher volume flow were to be passed through the irradiation device, it would not be possible to allow a sufficiently long dwell time in the irradiation chamber -provided that a purification device which is portable from an economic point of view is still to be used.

The invention thus enables economical and efficient cleaning of the medium flow passing through the purification device.

The irradiation device may be arranged in the guiding means and may preferably be formed by a section of the guiding means in which, in particular, at least one UV radiation source is arranged.

The inner side of the irradiation device and/or of the guiding means in the region of the irradiation device can in particular be reflective at least in certain areas, preferably over the entire surface, and in particular have a reflection factor for UV radiation of at least 0.6.

A radiation source can be provided in the irradiation device for irradiating the medium flowing through the irradiation device and/or the partial flow.

The interior of the irradiation device and/or the enclosed interior of the irradiation device and/or the guiding means can in particular be regarded as a UV treatment chamber for treating the partial flow and/or the partial flow of the medium conducted in through the irradiation device. Preferably, the inner wall is a reflector, which is further preferably formed as an inserted and/or replaceable housing component.

For the purposes of the present invention, the reflection factor is understood to be the ratio between reflected and incident intensity as a quantity of energy. In particular, the reflection factor may depend on the material of the inner wall on which the radiation impinges.

According to the invention, it is also possible to kill bacteria, viruses and/or fungi with a comparatively high UV resistance, in particular by ensuring the longest possible residence time in the irradiation device.

Thus, by increasing the intensity of UV radiation, the radiation dose can be increased, especially for the same duration of exposure of microorganisms to UV-C radiation.

In addition, the efficiency of UV disinfection is influenced by the resistance value of the microorganisms (k [m²/J]) to UV radiation, the exposure time (t [s]) and the intensity of the radiation (I [W/m²]). The resistance value k of the microorganisms cannot be influenced and can in particular be between 0.001 to 0.5 m²/J, preferably between 0.0014 to 0.3 m²/J.

According to the invention, it can be achieved that for a given UV resistance (k-factor), a low exposure time is sufficient by increasing the total intensity, since the variables ultimately influence each other in particular. Thus, both by increasing the exposure time and by increasing the intensity, the inactivation of the microorganisms after overcoming the UV resistance can be achieved.

In particular, the microorganism kill rate can be determined by the following formula:

$$S ( t ) = {S}_{o} {e}^{- I \cdot k \cdot t}$$

with:
I=intensity (W/m²),
k=UV rate of the microorganism (m²/J),
t=the dwell time (s)
S=microbial content
Sₒ=initial microbial content.

In particular, the purification device according to the invention can meet at least a six-fold ACH (air change per hour) required, for example, in classrooms, especially with a volumetric flow rate to be treated of approximately 1200 m³ per hour.

According to the invention, the purification device can be a mobile air disinfection device or one that can be mounted on at least one wall or ceiling. However, it may also be a stationary device.

The real separation-treatment volume of the treated microorganisms is decisive for the design of the purification device, which, according to the invention, can meet the legal requirements at a high total treated volume flow rate. This real separation-treatment volume rate is called the CADR (clean air delivery rate) and ultimately provides a means of comparison. For the CADR, main influencing factors are, for example, the treated volume flow Q through the purification device, the internal separation-treatment efficiency N when the air flow passes through, and the so-called Fluidal Efficiency Factor (dimensionless F) of the device-generated air flow in a room. The CADR can thus be described by the following functions:

$$CADR = Q [ {m}^{3} / h ] \cdot N [ \% ] \cdot F$$

If the required CADR is known, the treated air volume can thus be derived. The determination of the characteristic value F thus plays a major role from a design point of view.

According to the invention, in particular, a high treatment dose of the partial flow treated in the irradiation device can be made possible.

This is necessary, for example, to efficiently kill the corona virus.

Ultimately, according to the invention, a solution can be provided in which a high cleaning performance is achieved by splitting the flows, which unexpectedly is significantly better than if the total flow had been passed through the irradiation stunt device.

In a particularly preferred embodiment, it is provided that the purification device comprises a housing having an inlet and an outlet for the medium. In particular, the housing can be flowed through by the flow of the medium conducted through the purification device. Alternatively or additionally, it can be provided that the irradiation device is arranged in the housing and/or in the interior of the housing.

Preferably, the guiding means fluidically connects the inlet to the irradiation device. Alternatively or additionally, the at least one further guiding means can preferably be flow-connected to the inlet. In particular, the further guiding means is not flow-connected to the irradiation device, but is flow-connected to the inlet. The further guiding means can thereby depart from the guiding means or be merged with the guiding means—namely after the partial flow has passed the irradiation device. In this way, the guiding means can also be continued in the direction of flow downstream of the irradiation device and preferably also be connected to the outlet.

Accordingly, in particular, the further guiding means and/or the guiding means can fluidically connect the inlet to the outlet.

In a particularly preferred embodiment, it is provided that a plurality of further guiding means are provided for guiding a plurality of further partial flows. In particular, at least 3, preferably between 3 to 10, more preferably between 4 to 5, further guiding means are arranged in the purification device according to the invention. The further guiding means can in particular surround the irradiation device and/or be arranged around the irradiation device and/or next to it. In this context, it is understood that the further guiding means are designed in such a way that they do not open into the irradiation device and/or are guided past the irradiation device and/or are spatially separated and/or flow-separated from the irradiation device. In particular, the further guiding means are designed as bypass lines to the irradiation device. Accordingly, the partial flow guided through the further guide means has a flow path and/or a flow way which does not lead into or through the irradiation device, but is separated from the irradiation device (in terms of flow).

Preferably, the guiding means and/or the at least further guiding means, preferably all further guiding means, is designed as a, preferably closed, flow guiding channel. The flow guide channel can in particular be a tube. Ultimately, the guiding means and the further guiding means serve to guide the respective partial flow and to specify the relevant flow path of the respective partial flow.

In particular, the inlet may have a plurality of inlet openings, preferably for drawing in medium flow. Alternatively or additionally, it can be provided that the outlet has a plurality of outlet openings. Accordingly, the inlet and/or the outlet need not necessarily be formed by a single opening, but the entire area provided for sucking in (inlet) and/or discharging (outlet) the medium serves as inlet and/or outlet.

In a further embodiment, it is particularly preferred that at least one inlet opening is assigned to the guiding means and/or at least one further inlet opening is assigned to the at least one further guiding means. In particular, the at least one inlet opening assigned to the guiding means cannot be assigned to the further guiding means at the same time. Thus, the respective partial flow guided through the guiding means and through the further guiding means can be supplied to the purification device via different inlet openings.

Furthermore, at least one outlet opening may be associated with the guiding means and/or at least one further outlet opening may be associated with the at least one further guiding means. In particular, the outlet opening for the guiding means is not assigned to the further guiding means. Thus, different outlet openings can be provided depending on the guiding means. In this context, it is understood that the guiding means or the further guiding means can also be assigned a plurality of outlet openings for discharging the medium flow. The outlet openings or the inlet openings can be arranged adjacent to one another or spaced apart from one another and, in particular, can be designed as slots.

Preferably, an inlet means fluidly connected to the inlet, in particular an inlet channel and/or inlet tube, is provided. The inlet means can open into the guiding means and the at least one further guiding means. In particular, the inlet means may be branched into the guiding means and the at least one further guiding means. Valves can be arranged in the respective branches to control the volume flow. Thus, the guiding means and the further guiding means can branch off from the inlet means, controlled accordingly as required. The inlet means can be flow-connected to the inlet openings, and/or the inlet openings can merge into the inlet means.

Furthermore, in a further preferred embodiment, it is provided that an outlet means is flow-connected to the outlet. In particular, the outlet means can open into the outlet openings of the outlet. The outlet means may ultimately be formed as a common channel and/or common tube for the medium flow. The guiding means and/or the partial flow exiting the irradiation device may open into the outlet means. In addition, the at least one further guiding means may also open into the outlet means. In particular, the individual partial flows can be brought together in the outlet means and preferably mixed with one another and thus ultimately exit the purification device together. By mixing the partial flows, it is achieved in particular that a total medium stream exiting the purification device can be provided with a high efficiency of air purification.

Preferably, the purification device is designed in such a way that the partial flow exiting the irradiation device has a higher velocity than the at least one further partial flow which is guided in the at least one further guiding means and/or whose flow way is directed along the further guiding means. In particular, the partial flow exiting the irradiation device may have a higher velocity than the at least one further partial flow by at least 20%, preferably at least 30%, further preferably at least 40%, and/or by at least 1 m/s, preferably by at least 2 m/s. The higher velocity is accompanied by the advantageous fluidic effect that the partial flow exiting the irradiation device is set up in particular to “drag along” the at least one further partial flow and thus ultimately to provide a so-called “venturi drag flow”. In particular, the higher velocity means that no separate blower devices or the like are required to generate the further partial flow per se. The flow of the further partial flow can ultimately be ensured by the drag effect of the partial flow having the higher velocity and exiting the irradiation device.

Preferably, a filter device is arranged in the at least one further guiding means, preferably in each further guiding means. The filter device can be designed in particular as a so-called HEPA filter. Alternatively or additionally, it can be provided that the filter device is designed in such a way that particles with a diameter of greater than 1 μm, preferably greater than 0.5 μm, are filtered out of the medium flow. The HEPA filter is also referred to as a high-efficiency submicron particulate air filter, which can be designed to separate suspended particles from the air. Filter mats may be used in the filter device. In particular, reference may be made to the European standard for the classification of high-efficiency submicron particulate air filters, namely EN 1822-1:2009 (as of April 2021).

In addition, a prefilter can also be arranged upstream of the inlet and/or in the guiding means, preferably in the direction of flow upstream of the irradiation device and/or the section of the guiding means forming the irradiation device. In particular, the prefilter is designed as a HEPA filter. Alternatively or additionally, it can be provided that the prefilter is designed in such a way that particles with a diameter greater than 1 μm, preferably greater than 0.5 μm, are filtered out of the medium flow.

Thus, in particular, particles can be filtered out of the medium flow that would otherwise produce a so-called “shadow formation” in the UV treatment chamber during the resulting interaction with the UV radiation. Ultimately, it is relevant in this context that the UV radiation is in the order of magnitude between 0.2 to 0.3 μm. However, since the aforementioned particles, for example dust particles or pollen or the like, have a larger diameter than the wavelength, the wavelength in particular cannot pass through particles with a diameter greater than 1 μm. For example, a bacterium may have a diameter of approximately 0.3 μm. According to the invention, it has been found that the shadow effect is present in front of particles with a diameter smaller than 0.5 μm, in particular between 0.3 μm to 0.5 μm, but the achieved killing of the microorganisms can still be tolerated. Thus, those microorganisms with a diameter larger than the wavelength of the UV radiation can also be rendered harmless, since the UV radiation also hits them.

Pre-filtering has the advantage that, in particular, the irradiation device can be supplied with a partial flow that has already been subjected to a filter stage. Thus, efficient disinfection of the partial flow can take place in the irradiation device.

In further embodiment examples it can also be provided that in at least one further guiding means likewise a further irradiation device for UV irradiation, in particular for UV-C irradiation, of the further partial flow is arranged. The further irradiation device, which in particular has a further radiation source for emitting the UV radiation, can increase the efficiency and/or the overall degree of separation achieved of the microorganisms present in the medium stream. Ultimately, the purification of the partial flow guided in the further guiding means can be designed in particular at least substantially independently of the purification of the partial flow guided in the guiding means. An adaptation to different usage situations and/or specifications can be made accordingly. It can also be provided, for example, that two irradiation devices are provided in the purification device, one irradiation device being connected in terms of flow to the guiding means and a further irradiation device being connected in terms of flow to the further guiding means. In addition, further guiding means may be provided in which no irradiation device is arranged. Such an arrangement is particularly advantageous, for example, for the treatment of a high volumetric flow.

The further irradiation device may furthermore be formed by a section of the further guiding means in which the further radiation source is arranged. For further explanations of the further irradiation device, reference may be made to the embodiment for the irradiation device, which apply in the same way to the further irradiation device.

In a particularly preferred embodiment of the invention, it is provided that a blower device is provided for sucking in and/or blowing off the medium, in particular for generating the medium flow from the inlet to the outlet. The blower device may be associated with the guiding means and/or the further guiding means. Ultimately, the blower device ensures that the medium can flow through the purification device via the inlet. The blower device can be arranged at different locations with regard to the flow path of the medium flowing through the purification device. Thus, the blower device can be associated with either the inlet or the outlet. It is particularly preferred that the blower device is arranged in the region of the inlet and/or, with regard to the flow way of the partial flow, upstream of the irradiation device. In principle, however, it is also possible in further embodiments for the blower device to be arranged downstream of the irradiation device with respect to the flow path of the partial flow. The arrangement of the blower device can be selected in particular as a function of different customer requirements.

Preferably, the blower device comprises a fan for generating the partial flow in the guiding means from the inlet to the irradiation device, preferably to the outlet. In particular, the blower device may also comprise a plurality of fans. Thus, the blower device may also comprise at least one further fan configured to generate the at least one further partial flow, namely to generate the partial flow in the at least one further guiding means from the inlet to the outlet. The number of fans of the blower device may be selected depending on the volumetric flow rate of the medium flowing through the purification device.

Preferably, the blower device and/or the inlet means has an actuating means for controlling the volumetric flow of the partial flow guided into the guiding means. In addition, the blower device and/or the inlet means can have at least one further actuating means for controlling the volumetric flow of the at least one further partial flow guided into the at least one further guiding means. A valve and/or a butterfly valve may be provided as the actuating means and/or further actuating means. Thus, the actuating means in combination with the at least one further actuating means enables the volumetric flows of the respective partial flows to be controlled individually as a function of different specifications. For example, the partial flow supplied to the irradiation device can be adjusted with respect to the volumetric flow, in particular so that a sufficient dwell time in the irradiation device can be ensured. The further actuating means can also be designed to ensure that, in the case of a plurality of further guiding means, a supply of the respective further partial flows to the respective further guiding means is successful. Ultimately, the control means enable the medium flow to be separated into individual partial flows.

In particular, in the context of the present, the irradiation dose is at least 30 J/m², preferably at least 50 J/m², more preferably at least 70 J/m² and preferably 100 J/m^{2+/-20}%.

In particular, the irradiation device can be designed to treat a volumetric flow of the partial flow of at least 100 m³/h, preferably of at least 200 m³/h, further preferably between 300 to 600 m³/h. At the aforementioned volumetric flows, a sufficient dwell time with a sufficiently high irradiation dose for efficient killing of the microorganisms present in the medium flow is ensured by the irradiation device. However, the total volumetric flow rate may be higher than the partial flow rate passed through the irradiation device.

In this context, it is particularly preferred that the purification device is designed in such a way that the volumetric flow of the partial flow guided through the guiding means has a proportion of at least 10%, preferably between 20% and 80%, more preferably between 30% and 50%, of the total volumetric flow of the medium flow passing through the purification device. The above data make it clear that the total volumetric flow can be higher than the volumetric flow of the partial flow passing through the irradiation device. In particular, a volumetric flow of at least 800 m³/h, preferably of at least 1000 m³/h, more preferably between 1000 to 3000 m³/h, can be treated in the purification device.

It is particularly preferred if the further guiding means is arranged at least substantially parallel to the guiding means. As previously explained, the guiding means may be surrounded by the further guiding means. Preferably, the irradiation device may also be surrounded by the further guiding means. In this context, it is also understood that the outlet opening into which the further guiding means opens may be arranged on a different wall than the outlet opening into which the guiding means opens. However, it is preferred if the outlet opening for the guiding means and the further guiding means are arranged on the same wall of the housing and, in particular, adjacent to one another. In particular, it is preferred if the guiding means and the at least one further guiding means open into the same outlet opening.

In a further, particularly preferred embodiment, it is provided that a control device is provided for controlling, connecting, disconnecting and/or regulating the volumetric flow of the partial flow and/or of the at least one further partial flow. In particular, the control device is designed for controlling and/or regulating the actuating means and/or the at least one further actuating means and/or the blower device. By means of the control device, in particular the total volume flow which is guided through the purification device can be changed and/or adapted. At the same time, the partial flow guided through the irradiation device can also be adjusted by the control device.

Further, it is particularly preferred that the guiding means and/or the further guiding means and/or the further guiding means is at least substantially entirely arranged within the housing.

Preferably, the housing and/or the purification device has a length between 30 to 200 cm, preferably between 80 to 150 cm. The housing and/or the purification device may have a diameter, preferably an inner diameter, between 8 to 30 cm, preferably between 10 to 20 cm. In a particularly preferred embodiment, the housing and/or the purification device has a length between 80 to 150 cm and an inner diameter between 10 to 20 cm. With the aforementioned dimensions, efficient inactivation of the microorganisms can be ensured in particular for a medium flow with a flow velocity between 1 to 2 m/s.

Furthermore, the purification device according to the invention can also be used in air conditioning systems or office buildings or the like. It is understood that the purification device can be designed depending on the medium flow and/or the flow rate of the medium flow.

Furthermore, in another preferred embodiment, it may be provided that the radiation source comprises a plurality of illuminants, preferably LEDs. Furthermore, alternatively or additionally, the radiation source may have an at least substantially elongated and/or rod-shaped form. In particular, the radiation source may have a length of at least 5 cm, preferably between 5 cm to 30 cm, more preferably between 10 cm to 20 cm. In a particularly preferred embodiment, it is provided that the radiation source has an elongated shape with a plurality of illuminants, preferably LEDs, arranged along an “array”.

In addition, the radiation source may have a diameter of at least 1 cm, preferably between 1 cm and 20 cm, more preferably between 2 cm and 10 cm, and in particular between 5 cm+/−1 cm. In particular, the diameter of the radiation source may also depend on the diameter of the illuminant used in the radiation source.

Alternatively or additionally, the radiation source can also be designed as a UV low-pressure lamp, in particular a low-pressure mercury discharge lamp, and/or as a UV medium-pressure lamp.

Finally, the radiation source can provide the UV radiation required to inactivate the microorganisms, in particular the UV-C radiation. In this context, the radiation source can have an intensity at its surface of between 1000 and 8000 W/m², preferably between 2000 and 6000 W/m² and in particular of 4200 W/m^{2+/-20}%.

Furthermore, the radiation source can in particular provide a power of at least 100 W, preferably of 190 W+/−10%. The power in the UV-C radiation range may preferably be between 10 to 100 W, more preferably between 50 to 70 W. The radiation emitted by the radiation source can decrease with its intensity in the distance square.

In a further preferred embodiment of the invention, it is provided that a plurality of radiation sources is arranged in the irradiation device and/or the section of the guiding means forming the irradiation device. In particular, between 2 to 10, preferably between 2 to 5, radiation sources can be arranged in the irradiation device and/or the section of the guiding means forming the irradiation device. The radiation sources can each in particular also have a plurality of illuminants, preferably LEDs. The multiple radiation sources can provide radiation that is at least substantially uniform and/or sufficient to inactivate the microorganisms over the length of the irradiation device.

Furthermore, the radiation from the multiple radiation sources can also interact with each other in the resulting interference pattern in the UV treatment chamber and contribute to increasing the constructive interference. The suitability for constructive interference specified according to the invention applies in particular to each radiation source used in the irradiation device according to the invention.

In principle, it is also possible for the radiation sources to be designed differently from one another. Preferably, the radiation sources are designed to be at least substantially identical.

The medium flow and/or the respective partial flow can be guided at least essentially laminarly through the guiding means and/or the further guiding means. Alternatively or additionally, however, it can also be provided that the respective partial flow flowing through forms a turbulent flow.

Furthermore, at least one radiation source can emit UV radiation in a wavelength range of at least 240 to 300 nm, preferably in a wavelength range of 250 to 285 nm, more preferably of 270 to 280 nm and in particular of 254 nm+/−10% and/or of 278 nm +/−10%. In the case of UV-C radiation with a wavelength of 254 nm+/−10%, a high level of virus inactivation can be achieved in particular. According to the invention, UV radiation with a wavelength in the aforementioned order of magnitude enables the microorganisms in the medium stream to be killed.

Furthermore, the present invention relates to a method for inactivating microorganisms present in a gaseous medium, in particular air, flowing through a purification device of the aforementioned type. In particular, air is provided as the medium. The microorganisms in the medium can also be killed. Bacteria, germs, molds and/or viruses may be provided as microorganisms. In the method according to the invention, the medium flows through the purification device. Only a partial flow of the total flow of the medium flowing through the purification device as a whole is supplied to the irradiation device via the guiding means. The flow way of the partial flow begins in particular at the inlet and ends in the irradiation device and/or the outlet. At least one further partial flow is guided through the further guiding means. The further partial flow is in particular flowwise separated from the partial flow and/or is not guided through the irradiation device. Ultimately, the further partial flow can be regarded as a bypass flow which is guided past or around the irradiation device.

With regard to advantages and preferred embodiments, reference may be made to the preceding explanations concerning the purification device, which also apply in the same way to the method according to the invention.

The method according to the invention thus provides efficient inactivation of microorganisms present in the medium and thus enables efficient purification of the medium, in particular of air. According to the invention, the method has made it possible for air disinfection to be carried out in particular only for a partial flow of the entire medium flow.

Furthermore, it is understood that any intermediate intervals and individual values are included in the aforementioned intervals and range limits and are to be regarded as disclosed as essential to the invention, even if these intermediate intervals and individual values are not specifically indicated.

Further features, advantages and possible applications of the present invention will be apparent from the following description of examples of embodiments based on the drawing and the drawing itself. In this context, all the features described and/or illustrated constitute the subject-matter of the present invention, either individually or in any combination, irrespective of their summary in the claims or their relation back.

### BRIEF DESCRIPTION OF THE DRAWINGS

FIG. 1 is a schematic perspective view of a purification device according to the invention:

FIG. 2 is a schematic side view of a further embodiment of a purification device according to the invention:

FIG. 3 is a schematic principle sketch of a further embodiment of a purification device according to the invention:

FIG. 4 is a schematic principle sketch of a further embodiment of a purification device according to the invention:

FIG. 5 is a schematic principle sketch of a further embodiment of a purification device according to the invention;

FIG. 6 is a schematic principle sketch of a further embodiment of a purification device according to the invention:

FIG. 7 is a schematic principle sketch of a further embodiment of a purification device according to the invention:

FIG. 8 is a schematic principle sketch of a further embodiment of a purification device according to the invention:

FIG. 9 is a schematic principle sketch of a further embodiment of a purification device according to the invention;

FIG. 10 is a schematic perspective view of a further embodiment of a purification device according to the invention:

FIG. 11 is a schematic perspective view of a further embodiment of a purification device according to the invention:

FIG. 12 is a schematic perspective view of a further embodiment of a purification device according to the invention:

FIG. 13 is a schematic perspective view of a further embodiment of a purification device according to the invention; and

FIG. 14 is a schematic perspective view of a further embodiment of a purification device according to the invention.

### DETAILED DESCRIPTION

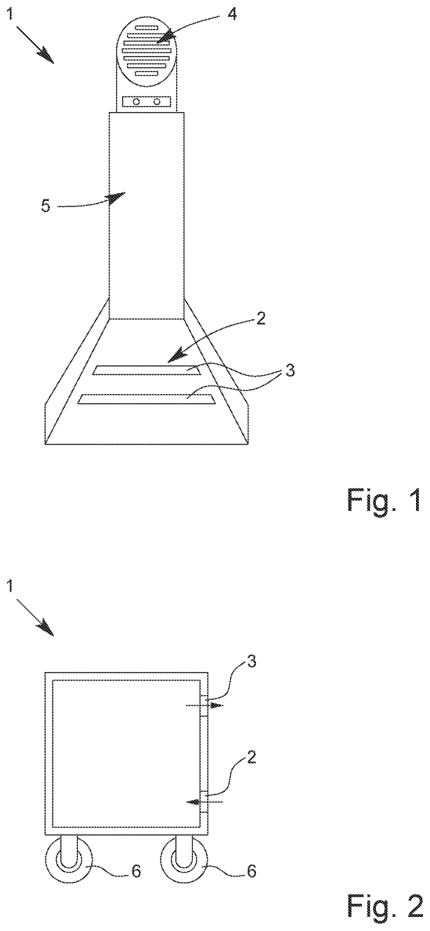

FIG. 1 shows a purification device 1 which is provided for inactivating and/or killing of microorganisms, the microorganisms being located in a gaseous medium flowing through the purification device 1. The medium flowing through the purification device 1 may be air and/or a gas mixture and/or a vapor mixture. The microorganisms to be inactivated may be, in particular, bacteria, germs, mold and/or viruses.

FIG. 1 shows that the purification device 1 is designed as a stand-alone device, in particular a mobile one, wherein the medium is air and is drawn into the purification device 1 via an inlet 2, which may have a plurality of inlet openings 3. The medium leaves the purification device 1 via an outlet 4, which may also have a plurality of outlet openings 12. Both the inlet 2 and the outlet 4 are arranged in and/or on a one-piece or multi-piece housing 5. The inlet 2 or the outlet 4 may each be arranged on at least one wall of the housing 5, or distributed on a plurality of walls.

FIG. 2 shows a mobile purification device 1, which can be moved and/or displaced, for example, via wheels 6. The medium is drawn into the purification device 1 via the inlet 2 and exits the purification device 1 via the outlet 4. The flow path is shown schematically by arrows.

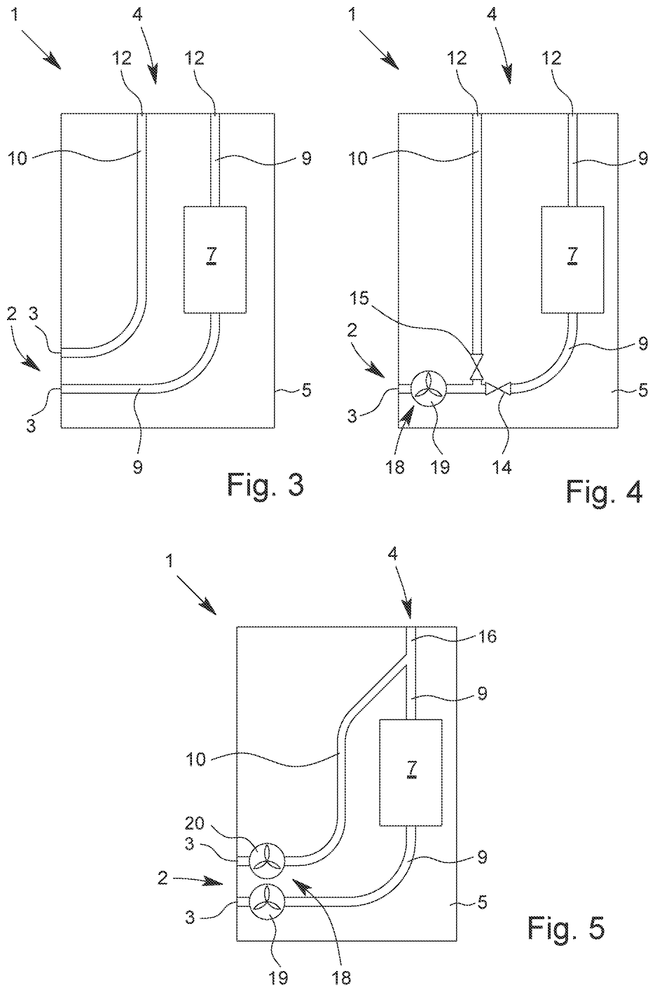

In FIG. 3, it is shown that the purification device 1 has an irradiation device 7. The irradiation device 7 is provided for UV irradiation, in particular UV-C irradiation, of the medium.

The irradiation device 7 may have at least one radiation source 8, as shown for example in FIG. 11, where two radiation sources 8 are shown in FIG. 11.

The radiation source 8 can emit UV radiation in a wavelength range of at least 240 nm to 300 nm, preferably in a wavelength range of 254 nm+/−10% and/or of 278 nm +/−10%. In principle, it can be provided that a plurality of radiation sources 8 are arranged in the irradiation device 7, in particular between 2 to 5. The radiation source 8 can have a diameter of at least 1 cm. A plurality of illuminants, preferably LEDs, may also serve as radiation source 8 in further embodiments. FIG. 11 shows a rod-shaped form of the radiation source 8, which in particular has a length of between 10 cm to 20 cm. The purification device 1 shown in FIG. 1 is arranged in particular in the housing 5, wherein the housing 5 can have a length and/or height between 80 to 150 cm.

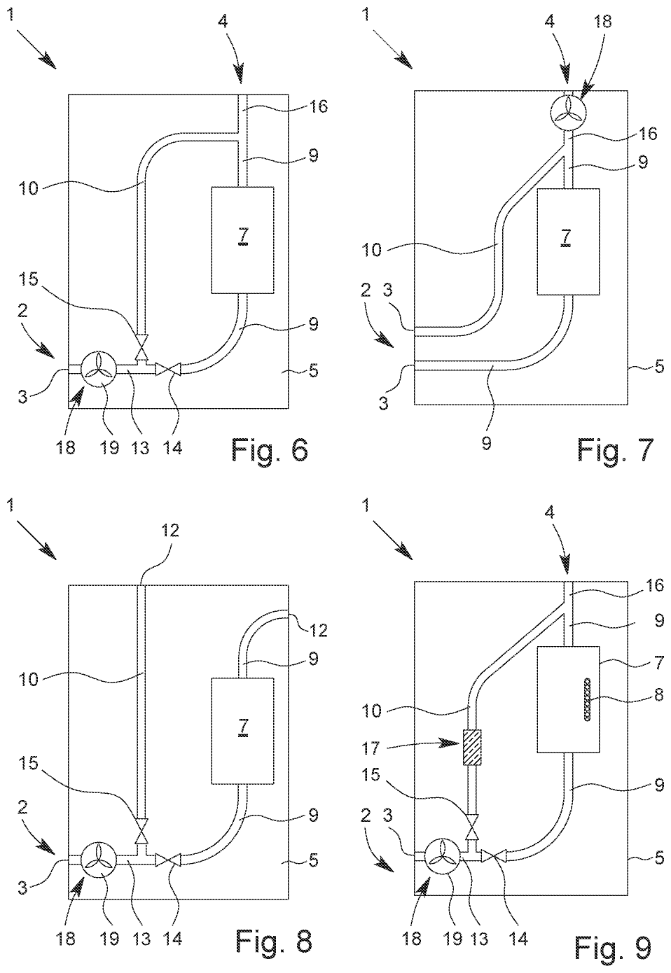

FIGS. 3 to 9 show principle sketches of the modes of operation of different embodiments of a purification device 1 according to the invention.

In FIG. 3, it is shown that a guiding means 9 is provided, wherein the guiding means 9 fluidically connects the irradiation device 7 to the inlet 3. The guiding means 9 serves to supply a partial flow of the medium to the irradiation device 7.

The irradiation device 7 can be designed as a separate component.

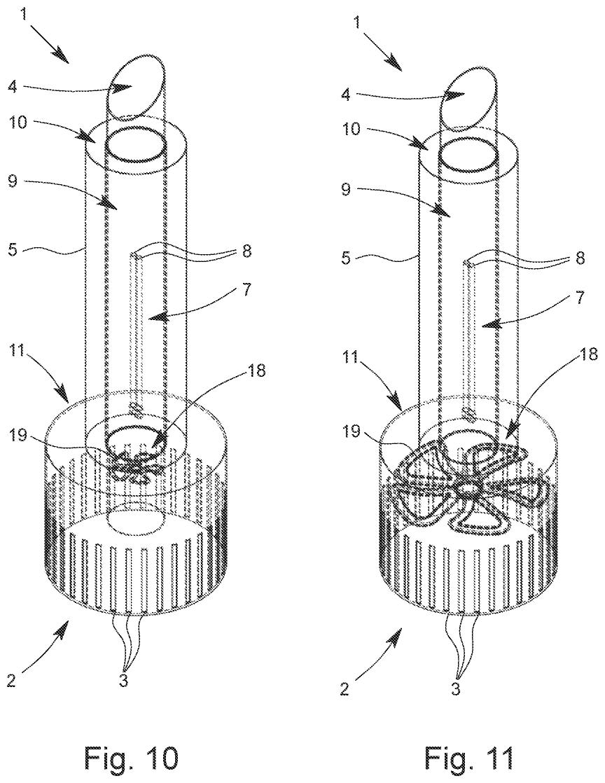

Alternatively, it is possible that the irradiation device 7 is formed by a section of the guiding means 9 in which the at least one radiation source 8 is arranged. This is shown schematically in FIGS. 10 to 14.

In particular, the guiding means 9 is arranged inside the housing 5.

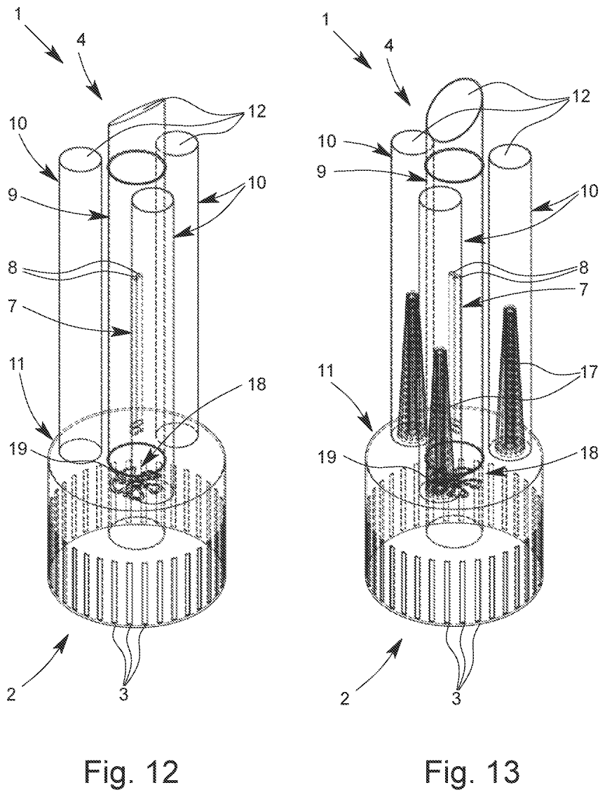

In addition to the guiding means 9, the purification device 1 shown in FIG. 3 has a further guiding means 10. In the embodiment examples shown in FIGS. 3 to 9, only one further guiding means is provided. In principle, a plurality of further guiding means can also be used in the purification device 1, as shown schematically in FIG. 12, for example. In particular, between 2 to 5 further guiding means 10 can be provided. The further guiding means 10 serve to guide a further partial flow of the medium. The further partial flow is not guided through the irradiation device 7 and/or the flow path of the further partial flow does not run through the irradiation device 7. The further guiding means 10 also does not open into the irradiation device 7.

FIG. 3 shows that the further guiding means 10 is guided past the irradiation device 7 and is also spatially separated from the irradiation device 7 and/or is not directly connected to the irradiation device 7. Also, the further partial flow guided in the further guiding means 10 is separated in terms of flow from the partial flow guided in the guiding means 9.

In FIG. 4, it is shown that the further guiding means 10 is ultimately designed as a bypass which is guided past the irradiation device 7.

In the embodiment examples shown in FIGS. 10 and 11, it is provided that the guiding means 9 is surrounded and/or enclosed by the further guiding means 10. Ultimately, however, the further partial flow guided by the further guiding means 10 is independent of the partial flow guided in the guiding means 9 and supplied to the irradiation device 7. In the embodiment shown in FIG. 11, too, at least two partial flows are provided which are separated from one another in terms of flow.

FIG. 5 shows that these partial flows can be recombined before being fed to the outlet 4. At least in the area (viewed in the direction of flow) of the irradiation device 7, the partial flow guided in the guiding means 9 is fluidically and/or flow-wise independent and/or separate from the further partial flow guided in the further guiding means 10.

Accordingly, in the embodiment example shown in FIG. 3, only a partial flow of the medium flowing through the purification device 1 is supplied to the irradiation device 7 via the guiding means 9. The remaining flow of the medium flowing through the purification device 1 is guided through the at least one further guiding means 10.

In addition, FIG. 3 shows that the further guiding means 10 is separated from the guiding means 9 in terms of flow, wherein the further guiding means 10 connects the inlet 2 to the outlet 4 in terms of flow. In the embodiment examples shown in FIGS. 3 and 4, it is provided that the guiding means 9 guides the partial flow to the outlet 4 after exit from the irradiation device 7. The guiding means 9 goes at least as far as the supply to the irradiation device 7.

According to the invention, for example, the partial flow passed through the irradiation device 7 can have a volumetric flow of 450 m³/h+/−10%, which can be mixed with a further partial flow of about 200 m³/h+/−20%. During the first pass through the purification device 1, a separation of the harmful microorganisms of at least 95%, preferably at least 98%+/−2%, can be achieved in particular.

FIG. 12 shows that the guiding means 9 and the at least one further guiding means 10 are designed as a closed flow guiding channel. In the embodiment example shown in FIG. 12, a tube in particular is provided as the guiding means 9 and as the further guiding means 10. The irradiation device 7 can also be arranged with its radiation source 8 inside the guiding means 9. The medium flowing through the irradiation device 7 is disinfected, in particular by means of UV treatment.

In the embodiment example shown in FIG. 10, it is provided that the inlet 2 is arranged in the region of a base 11 of the purification device 1. The base 11 may ultimately be designed as a pedestal. Circumferentially, a plurality of inlet openings 3 are provided around the base 11, which are spaced apart from one another and, in the embodiment example shown in FIG. 10, are designed in particular in the shape of a slit. In the embodiment example shown in FIG. 10, the inlet openings 3 are provided for and/or assigned to both the guiding means 9 and the further guiding means 10.

In the embodiment example shown in FIG. 3, it is provided that one inlet opening 3 each is provided for the guiding means 9 and the further guiding means 10. It is not shown that a plurality of inlet openings 3 can also be provided for the guiding means 9 and the further guiding means 10. In particular, the inlet opening 3 assigned to the guiding means 9 cannot be assigned to the further guiding means 10 at the same time.

In FIG. 4, it is shown that the outlet 4 has a plurality of outlet openings 12. In the example embodiment shown in FIG. 4, one outlet opening 12 is associated with the guiding means 9 and another outlet opening 12 is associated with the further guiding means 10. The outlet openings 12 can be arranged and formed in such a way that they are each associated with only one guiding means 9, 10. The outlet openings 12 and/or the inlet openings 3 can basically be arranged on the same wall of the housing 5 or on different walls.

In FIG. 8, it is shown that the outlet opening 12 of the guiding means 9 is arranged on a side wall, whereas the outlet opening 12 of the further guiding means 10 is arranged on an upper side of the housing 5.

FIG. 8 shows that an inlet means 13 is provided. The inlet means 13 can be associated in particular with the inlet 2 and at least one inlet opening 3. In the embodiment example shown in FIG. 8, the inlet means 13 is ultimately designed as an inlet channel, from which the guiding means 9 and the further guiding means 10 can be branched off. FIG. 8 shows that actuating means 14, 15 are provided for controlling and feeding the respective partial flow. The actuating means 14, 15 can be designed as butterfly valves or, as shown schematically in FIG. 8, as valves. The actuating means 14, 15 can be provided for controlling the volumetric flow of the partial flow fed into the guiding means 9 and/or of the further partial flow fed into the further guiding means 10.

FIG. 5 shows that an outlet means 16 is provided. The outlet means 16 opens into the outlet 4 and in particular into at least one outlet opening 12. The outlet means 16 can be designed as a channel. In the embodiment example shown in FIG. 5, it is provided that the guiding means 9 and the further guiding means 10 open into the outlet means 16. In particular, a mixing and/or merging of the partial flow and the at least one further partial flow can be provided in the outlet means 16. In terms of flow, the outlet means 16 is arranged downstream of the irradiation device 7—as seen in the direction of flow.

It is not shown that the purification device 1 can be designed in such a way that the partial flow leaving the irradiation device 7 has a velocity at least 20%, preferably at least 30%, higher than the further partial flow. This increased velocity can be used in particular to ensure that a sufficiently high velocity of the volumetric flow of the further partial flow is achieved in the further guiding means 10. For example, FIG. 12 shows that the flow present in the further guiding means 10 may ultimately be made possible by the flow of the partial flow passing through the irradiation device 7.

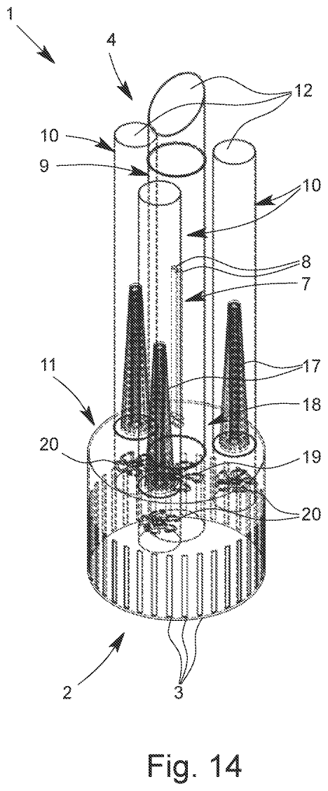

FIG. 9 shows schematically that a filter device 17 is arranged in the further guiding means 10. Filter devices 17 are furthermore also provided in the embodiment examples shown in FIGS. 13 and 14. A HEPA filter, for example, can be provided as the filter device 17. The filter device 17 can be designed in such a way that particles with a diameter greater than 0.5 μm are filtered out of the respective partial flow passed through the filter device 17.

It is not shown that a pre-filter is arranged upstream of the inlet 2 and/or in the guiding means 9, in particular wherein the pre-filter is designed in such a way that particles with a diameter greater than 0.5 μm are filtered out of the medium flow.

In FIGS. 4 to 9, it is shown schematically that a blower device 18 is used. The blower device 18 can have at least one fan 19. In the embodiment example shown in FIG. 5, it is provided that the blower device 18 has a further fan 20 in addition to the fan 19. The fan 19 can be designed for sucking in and for blowing off the partial flow that is guided through the irradiation device 7.

In addition, the fan can also be used to generate the further partial flow that is routed from inlet 2 to outlet 4. This is provided, for example, in the embodiment example shown in FIG. 11.

FIG. 5 shows that the blower device 18 has a further fan 20 for generating the further partial flow from the inlet 2 to the outlet 4. Further fans 20 are also provided in the embodiment example shown in FIG. 14.

In the embodiment examples according to FIGS. 10, 12 and 13, the blower device 18 has a fan 19 which is provided for the guiding means 9 and ultimately for generating the partial flow supplied to the irradiation device 7. However, due to the Venturi effect and/or fluidic effects, the increased velocity of the partial flow exiting the irradiation device 7 can also ensure a sufficiently high volume flow of the at least one further partial flow.

FIG. 7 shows that the blower device 18 with a fan 19—viewed in the direction of flow—is arranged behind the irradiation device 7.

However, the further embodiments make it clear that the blower device 18 is preferably arranged—as seen in the direction of flow—upstream of the irradiation device 7 and is preferably assigned at least to the guiding means 9.

Moreover, the actuating means 14, 15 may be associated with the blower device 18 and/or coupled to the blower device 18.

Not shown is that the irradiation device 7 is designed to treat a volumetric flow of the partial flow of at least 200 m³/h. In addition, the purification device 1 can be designed in such a way that the volume flow of the partial flow guided through the guiding means 9 has a proportion of at least 10%, preferably between 30 to 50%, of the total volumetric flow of the medium flowing through the purification device 1.

In FIGS. 12 to 14, it is shown that the further guiding means 10 are arranged at least substantially parallel to the guiding means 9 and are preferably spaced apart from the guiding means 9. In this case, the guiding means 9 can be surrounded by the further guiding means 10 and/or enclosed by them. In the embodiment examples according to FIGS. 10 and 11, it is shown that the guiding means 9 is ultimately arranged within the further guiding means 10 in such a way that the flow path and/or flow path of the partial flow guided through the irradiation device 7 is in particular independent of the flow way and/or flow path of the further partial flow. Ultimately, in the embodiment examples according to FIGS. 10 and 11, the further guiding means 10 is the intermediate space between the channels and/or tubes shown.

It is not shown that a control device is provided for controlling, connecting, disconnecting and/or regulating the volumetric flow, the partial flow and/or the at least one further partial flow. In particular, the control device not shown in more detail can be designed for controlling and/or regulating the actuating means 14 and/or the further actuating means 15.

In particular, the guiding means 9 and the at least one further guiding means 10 are arranged at least substantially completely in the housing 5.

### LIST OF REFERENCE SIGNS

1 Purification device
2 Inlet
3 Inlet opening
4 Outlet
5 Housing
6 Wheels
7 Irradiation device
8 Radiation source
9 Guiding means
10 Further guiding means
11 Base
12 Outlet opening
13 Inlet means
14 Actuating means
15 Further actuating means
16 Outlet means
17 Filter device
18 Blower device
19 Fan
20 Further fan

1. A purification device for inactivating microorganisms, such as bacteria, germs, mold and/or viruses, present in a gaseous medium, in flowing through the purification device, with an irradiation device—for UV irradiation of the medium, wherein a radiation source is provided in the irradiation device for irradiating the medium and/or the partial flow flowing through the irradiation device, wherein at least one radiation source is provided emitting UV radiation in a wavelength range from 250 to 285 nm,
wherein a guiding means flow-connected to the irradiation device is provided for supplying a partial flow of the flow of the medium flowing through the purification device to the irradiation device,
wherein at least one further guiding means is provided for guiding a further partial flow and wherein the further guiding means does not open into the irradiation device and/or is guided past the irradiation device and/or is spatially separated and/or flow-separated from the irradiation device.

2. The purification device according to claim 1, wherein a housing having an inlet and an outlet for the medium is provided, wherein the housing can be flowed through by the flow of the medium conducted through the purification device and/or wherein the irradiation device is arranged in the housing.

3. The purification device according to claim 2, wherein the guiding means fluidically connects the inlet to the irradiation device and/or wherein the further guiding means is fluidically connected to the inlet;
wherein the further guiding means fluidically connects the inlet to the outlet and/or wherein the guiding means fluidically connects the inlet to the outlet.

4. The purification device according to claim 1, wherein a plurality of further guiding means is provided for conducting a plurality of further partial flows.

5. The purification device according to claim 2, wherein the inlet comprises a plurality of inlet openings and/or wherein the outlet has a plurality of outlet openings;
wherein at least one inlet opening is associated with the guiding means and/or at least one further inlet opening is associated with the at least one further guiding means; and/or
wherein at least one outlet opening is associated with the guiding means and/or at least one further outlet opening is associated with the at least one further guiding means.

6. The purification device according to claim 2, wherein an outlet means is provided which is flow-connected to the outlet, wherein the guiding means and/or the partial flow exiting from the irradiation device and the at least one further guiding means open into the outlet means for mixing the partial flow exiting from the irradiation device and the at least one further partial flow.

7. The purification device according to claim 1, wherein the purification device is designed in such a way that the partial flow emerging from the irradiation device has a higher velocity, by at least 20%, than the at least one further partial flow.

8. The purification device according to claim 1, wherein a filter device is arranged in the at least one further guiding means and wherein the filter device is designed in such a way that particles with a diameter greater than 1 μm are filtered out of the medium flow.

9. The purification device according to claim 2, wherein a prefilter is arranged upstream of the inlet and/or in the region of the inlet and/or in the guiding means, wherein the prefilter is designed in such a way that particles with a diameter greater than 1 μm are filtered out of the medium flow.

10. The purification device according to claim 2, wherein a blower device is provided for sucking in and/or blowing out the medium.

11. The purification device according to claim 10, wherein the blower device comprises a fan for generating the partial flow in the guiding means from the inlet to the irradiation device, to the outlet, and/or wherein the blower device comprises at least one further fan for generating the at least one further partial flow in the at least one further guiding means from the inlet to the outlet.

12. The purification device according to claim 10, wherein the blower device has an actuating means for controlling the volumetric flow of the partial flow guided into the guiding means and at least one further actuating means for controlling the volumetric flow of the at least one further partial flow guided into the at least one further guiding means.

13. The purification device according to claim 1, wherein the irradiation device is designed to treat a volumetric flow of the partial flow of at least 100 m³/h.

14. The purification device according to claim 1, wherein the purification device is designed in such a way that the volumetric flow of the partial flow guided through the guiding means has a proportion of at least 10% of the total volumetric flow of the medium flow flowing through the purification device.

15. The purification device according to claim 12, wherein a control device is provided for controlling, connecting, disconnecting and/or regulating the volumetric flow of the partial flow and/or of the at least one further partial flow, the control device being designed for controlling and/or regulating the actuating means and/or the at least one further actuating means.

16. The purification device according to claim 7, wherein the purification device is designed in such a way that the partial flow emerging from the irradiation device has a higher velocity, by at least 50%, than the at least one further partial flow.

17. The purification device according to claim 8, wherein the filter device is designed in such a way that particles with a diameter greater than 0.5 μm are filtered out of the medium flow.

18. The purification device according to claim 12, wherein the actuating means is a valve and/or a butterfly valve and the at least one further actuating means is a valve and/or a butterfly valve.

19. The purification device according to claim 13, wherein the irradiation device is designed to treat a volumetric flow of the partial flow of between 300 to 600 m³/h.

20. The purification device according to claim 14, wherein the purification device is designed in such a way that the volumetric flow of the partial flow guided through the guiding means has a proportion of between 30% and 50% of the total volumetric flow of the medium flow flowing through the purification device.
